(19) United States
(12) Patent Application Publication
(10) Pub. No.: US 2019/0389940 A1
(43) Pub. Date: Dec. 26, 2019

(54) CONSTRUCTION AND APPLICATION OF RECOMBINANT GENE FOR CHIMERIC ANTIGEN RECEPTOR FOR TREATING HIV INFECTION

(71) Applicant: WUHAN BIO-RAID BIOTECHNOLOGY CO., LTD., Wuhan City, Hubei (CN)

(72) Inventors: Tongcun ZHANG, Wuhan City (CN); Chaojiang GU, Wuhan City (CN); Xinghua LIAO, Wuhan City (CN)

(73) Assignee: WUHAN BIO-RAID BIOTECHNOLOGY CO., LTD., Wuhan City, Hubei (CN)

(21) Appl. No.: 16/317,514

(22) PCT Filed: Aug. 28, 2017
(86) PCT No.: PCT/CN2017/099261

§ 371 Date: Jan. 11, 2019

(30) Foreign Application Priority Data
Jun. 28, 2017 (CN) 201710507860.8

(51) Int. Cl. C07K 16/10; C12N 15/86; C07K 14/705; C07K 14/725; C12N 5/0783; A61K 35/17; A61P 31/18

(52) U.S. Cl. CPC C07K 16/1063; A61K 2039/505; C07K 14/70517; C07K 14/70521; C07K 14/70578; C07K 14/7051; C12N 5/0636; A61K 35/17; A61P 31/18; C12N 2740/15043; C07K 2319/30; C07K 2319/33; C07K 2319/03; C07K 2319/02; C07K 2317/622; C07K 2317/76; C12N 15/86

(57) ABSTRACT

Relating to immunotherapy of infectious diseases, in particular to construction and application of a recombinant gene for chimeric antigen receptor (CAR) for treating HIV infection, a single-chain antibody ScFv is capable of recognizing gp120 on the surface of an HIV virus-infected cell, which is obtained by tandemly ligating light chain and heavy chain variable regions of an antibody against gp120 on the surface of the HIV virus-infected cell. The single-chain antibody is made into a chimeric antigen receptor (CAR), the CAR encoding gene is transferred into a plasmid vector, and the lentiviral vector into which the CAR encoding gene is transduced is transduced into CD8⁺ T lymphocytes. The obtained CAR-T lymphocytes have been found to have significant activity in inhibiting and killing HIV-infected cells in both in vitro and in vivo experiments, which can be used as an active ingredient for preparing a drug against HIV infection.

### TECHNICAL FIELD

The invention relates to the technical field of immunotherapy of infectious diseases, in particular to construction and application of a recombinant gene for a chimeric antigen receptor for treating HIV infection.

The sequence listing disclosed herein is included in a text file having the name “B1272-10033U01_SEQUENCE_LISTINGx,” created on Sep. 13, 2019, having a size of 54,000 bytes. The foregoing text file is incorporated herein by reference.

### BACKGROUND ART

AIDS is a major infectious disease that threatens the safety of human life caused by human immunodeficiency virus type 1 (HIV-1) infection. According to the latest statistics of the World Health Organization, more than 39 million people have died of AIDS since it was discovered by the end of 2014, and 37 million people are still living with HIV worldwide. The number of people living with HIV in China has increased year by year, and the total number of patients has exceeded one million. There is currently no effective vaccine, and existing drugs cannot completely cure AIDS.

Highly active antiretroviral therapy (HAART) is the first revolution in the history of HIV/AIDS treatment, which greatly reduces the incidence and mortality of HIV/AIDS, significantly prolongs patient life and even reduces HIV transmission. However, there are also many challenges: 1) patients must take their medicines for life, which requires expensive economic costs; 2) serious toxic and side effects; 3) emergence of drug-resistant strains of HIV; and 4) more importantly, cART cannot completely clear the virus, mainly because the drug is only effective against the virus in the replication, and is ineffective for the latent virus “reservoir” established by HIV in the early stages of infection. Once the antiretroviral therapy is interrupted, the integrated provirus in the virus reservoir is reactivated and viremia will rebound rapidly in almost all patients.

The high variability of HIV virus in its transmission and reproduction has made the previous effective AIDS drugs ineffective, therefore, “cocktail therapy” came later, which is to combine multiple antiviral drugs for HAART. However, cocktail therapy also has its limitations. In light of the current frontier and hotspot in the international research on AIDS, researchers generally believe that the main reason why HIV cannot be cured is that HIV establishes a hidden virus “reservoir” in the body at the very early stage of infection, that is, Resting memory CD4+ T lymphocyte.

One of the major challenges in finding a cure for AIDS is how to reactivate the latent HIV to be recognized and shuffled by the body's immune system (shock and kill). At the same time, the study found that even in the body of infected patients receiving cART therapy, there is also irreversible immune damage, especially the reduction of the number of cytotoxic T lymphocyte (CTL) and functional defects, indicating that the immune system reconstructed by antiretroviral therapy will not effectively clear those activated cells, and it is necessary to enhance the clearance of HIV reservoirs by a method of jointly enhancing the body's HIV-specific immune response. Therefore, the exploration of the formation and activation strategies of the HIV latent virus “reservoir” and the reconstruction of the body's immune function are all extremely valuable research directions in the era of curing the HIV.

In recent years, chimeric antigen receptor (CAR)-based tumor immunotherapy technology has created a new way to kill tumor cells. Due to its high affinity and MHC (Major histocompatibility complex) independence and other advantages, especially the third-generation CAR formed by coupling two CD28 and 4-1BB, and CD3ζ can enhance the tumoricidal ability of T cells and prolong their survival time in vivo, thus achieving encouraging results in tumor immunotherapy such as leukemia and lymphoma, etc. In fact, as early as 1994, Roberts et al. tried to treat HIV infection with CAR-T cells. They selected a CD4 sequence as a single-chain antibody for binding to gp120 on the surface of the infected cells. Although it had the function of partially killing infected cells and after years of efforts, it ended in failure. The main reasons are as follows: 1. the transduction efficiency using a retroviral vector is low, in order to obtain sufficient CAR-T cells for reinfusion, excessive expansion in vitro results in cell death and loss of CAR molecules after reinfusion; and 2. the CAR molecular design itself has defects, in which the CD4 domain may cause transduced CTLs to be infected by HIV, or the virus-infected cells may escape the killing of CAR-T cells by down-regulating the expression of CD4 molecules.

### Technical Problem

In order to overcome the above-mentioned deficiencies of the prior art, the present invention provides a novel single-chain antibody ScFv capable of recognizing gp120 on the surface of an HIV virus-infected cell and a chimeric antigen receptor (CAR) made of the single-chain antibody, which is called an N6-CAR molecule.

Another object of the present invention is to provide an expression vector capable of expressing the above N6-CAR and an N6-CAR vector gene-modified CD8⁺ T lymphocyte.

Still another object of the present invention is to provide a use of the N6-CAR molecule-modified CD8⁺ T lymphocyte for the preparation of a medicament against HIV infection.

### Technical Solution

In order to achieve the above objects, the present invention is achieved by the following technical solutions:

providing a single-chain antibody ScFv, which is capable of recognizing gp120 on the surface of an HIV virus-infected cell, is obtained by tandemly ligating light chain and heavy chain variable regions of an antibody against gp120 on the surface of the HIV virus-infected cell, and serves as an extracellular binding domain of the entire CAR molecule, and its amino acid sequence is shown in SEQ ID NO. 2.

The present invention also provides an encoding gene encoding the above single-chain antibody ScFv, the nucleotide sequence of which is shown in SEQ ID NO. 1.

The present invention also provides a chimeric antigen receptor for treating HIV infection, which is obtained by sequentially splicing a signal peptide, the single-chain antibody ScFv provided by the present invention, CD8 hinge, transmembrane domain of cluster of differentiation CD28-TM and intracellular domain ICD, 4-1BB and ζ chain of cluster of differentiation 3 CD3 from N-terminal to C-terminal, and the amino acid sequence of the obtained chimeric antigen receptor is as shown in SEQ ID NO. 4.

The present invention also provides an encoding gene encoding the above chimeric antigen receptor N6-CAR, the nucleotide sequence of which is shown in SEQ ID NO. 3.

An expression vector containing and capable of expressing an encoding gene of the chimeric antigen receptor N6-CAR having the amino acid sequence of SEQ ID NO. 4, the vector is a PTK-EF1α-N6 vector obtained by transformation by using a PTK881 vector as a backbone and replacing a CMV promoter with an EF1α promoter, the nucleotide sequence of which is shown in SEQ ID NO. 5.

The present invention provides a genetically modified CD8⁺ T lymphocyte, which is a genetically engineered T-lymphocyte capable of expressing a chimeric antigen receptor obtained by transducing a lentiviral vector of a 293T cell transfected with the PTK-EF1α-N6 expression vector into a CD8⁺ T lymphocyte. The N6-CAR-modified CD8⁺ cells have a more significant killing effect on cell lines expressing gp120.

Further, the genetically modified CD8⁺ T lymphocyte is prepared by the following methods: (1) isolating PBMC from peripheral blood and then using magnetic beads for positive screen to obtain CD8⁺ T cells, stimulating with anti-CD3/28 magnetic beads (ratio of cell to magnetic bead: 1:3) for 12 hours, adding a lentivirus recombinant with a N6-CAR molecule for infection for 4 hours, and then supplementing fluid (MOI=5); starting from the third day after virus infection, counting cells and supplementing the culture medium according to cell state and proliferation, adjusting the cell concentration to 0.6×10⁶/ml, and supplementing 100 U/mL IL-2; and further expanding the cells until the number of cells for reinfusion is met.

The present invention also provides a use of the above N6-CAR gene-modified CD8⁺ T lymphocyte, and the N6-CAR gene-modified CD8⁺ T lymphocyte is used to prepare a live cell drug against HIV infection.

### SUMMARY OF THE INVENTION

Beneficial effect of the present invention: (1) the present invention overcomes the shortcomings of the early design, utilizes a broad-spectrum neutralizing antibody capable of highly specifically binding to the viral protein Gp120 as a ScFv, and can bind to 98% of HIV-1 virus strains, thereby increasing the broad spectrum of the CAR-T cells; (2) the present invention uses a PTK plasmid containing a SIN (self-inactivating) structure to produce a lentiviral vector with increased safety, while modifying a CAR molecule into a bi-stimulatory molecule to increase the expansion and survival characteristics of N6-CAR-T cells, thereby increasing clinical efficacy and safety; and (3) the genetically modified CD8⁺ T lymphocyte capable of expressing a chimeric antigen receptor in the present invention has been found to have significant activity for inhibiting and killing HIV virus in both in vitro and in vivo experiments, and is capable of producing an anti-HIV infection drug as an active ingredient.

### BRIEF DESCRIPTION OF THE DRAWINGS

FIG. 1 is a schematic diagram showing the structure of the chimeric antigen receptor against HIV infection constructed by the present invention;

FIG. 2 is a schematic diagram showing the structure of the PTK-EF1α-N6 lentiviral vector constructed by the present invention;

FIG. 3 shows the detection of the expression level (A) of N6-CAR in the transduced CD8⁺ T lymphocytes and its functional proliferative potential (B) after stimulation in the present invention;

FIG. 4 shows the detection of the activity of the N6-CAR transduced CD8⁺ T lymphocytes of the present invention in killing HIV-infected cells in vitro;

FIG. 5 shows the detection of the activity of the N6-CAR transduced CD8⁺ T lymphocytes of the present invention in inhibiting virus under co-culture conditions; and

FIG. 6 shows the detection of the activity of the N6-CAR transduced CD8⁺ T lymphocytes of the present invention in killing HIV-infected cells in humanized mice.

### DETAILED DESCRIPTION OF THE INVENTION

The examples are shown to illustrate certain embodiments of the present invention and are not to be construed as limiting the scope of the present invention. The disclosure of the present invention can be modified from the materials, methods, and reaction conditions, all of which are within the spirit and scope of the present invention.

### Example 1

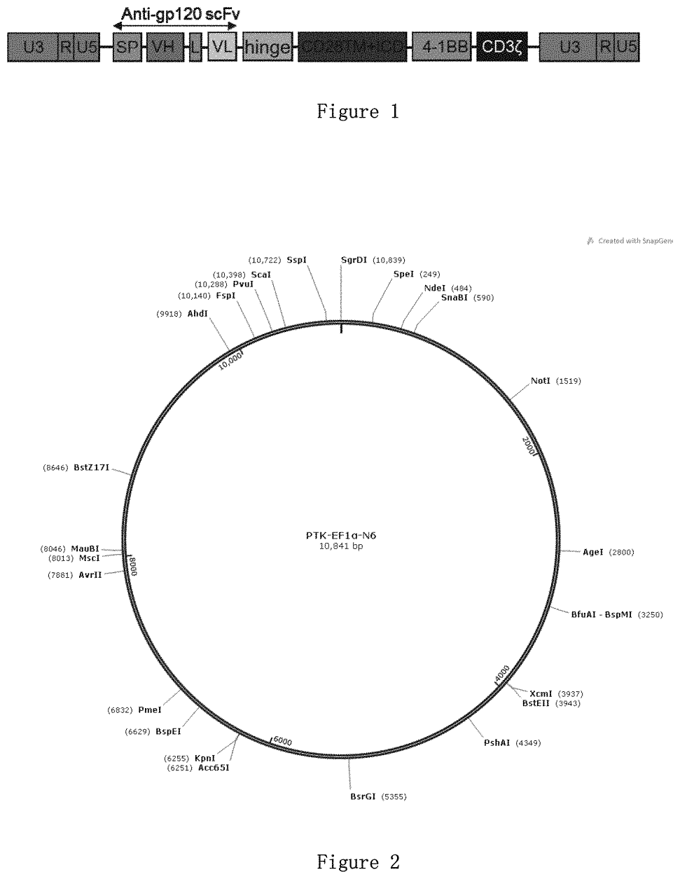

The present invention provides a chimeric antigen receptor (CAR) recombinant gene for treating HIV infection and a construction method thereof, and the specific splicing method is: sequentially splicing a signal peptide, a single-chain antibody ScFv capable of recognizing gp120 on the surface of an HIV virus-infected cell, CD8 hinge, transmembrane domain of cluster of differentiation CD28-TM+ICD, 4-1BB and ζ chain of CD3 (cluster of differentiation 3), and finally obtaining a complete chimeric antigen receptor (CAR) molecule capable of treating HIV, the amino acid sequence of which is SEQ ID NO. 4, and the structure of which is shown in FIG. 1; and the nucleotide sequence of a gene encoding the chimeric antigen receptor (CAR) is shown in SEQ ID NO. 3.

The amino acid sequence of the chimeric antigen receptor-derived single-chain antibody ScFv for treating HIV infection is shown in SEQ ID NO. 2, and the single-chain antibody is obtained by tandemly ligating light chain and heavy chain variable regions of an antibody against gp120 on the surface of the HIV virus-infected cells, and the nucleotide sequence of a gene encoding the same is shown in SEQ ID NO. 1.

The structural design of the CAR molecule of the present invention is described in detail by the chimeric antigen receptor (CAR) molecule shown in SEQ ID NO. 4. The N-terminal of the sequence of the CAR molecule is a CAR-derived ScFv sequence, which can specifically recognize gp120 on the surface of the HIV virus-infected cells; and the C-terminal of the sequence of the CAR molecule is based on a third-generation CAR structure, comprising CD8 hinge, CD28TM+ICD, 4-1BB and CD3ζ intracellular domain which are tandemly ligated. The ScFv and intracellular signal molecule are linked by the transmembrane domain of the CD28 molecule. In the above structure, each fragment can function as follows: the signal peptide can secrete CAR into the extracellular, the CD28TM+ICD anchors the CAR of the present invention to the cell membrane; the ScFv specifically recognizes gp120 on the surface of the HIV virus-infected cells; and CD3 is an intracellular signal activating sequence, which activates a signal after the ScFv binds to the antigen, and initiates killing activity of lymphocyte.

### Example 2: Recombinant Construction of PTK-EF-1α-N6 Plasmid Expression Vector with CAR Molecule

The CAR was synthesized according to the sequence shown in SEQ ID NO. 4, and the full-length CAR encoding gene was inserted into the target expression vector by a seamless recombinant cloning technique (see FIG. 2). After several experiments, the preferred plasmid vector was a PTK-EF1α-N6 vector (the nucleotide sequence of which is shown in SEQ ID NO. 5) obtained by transformation by using a PTK881 vector as a backbone and replacing the CMV promoter with the EF1a promoter. Finally a recombinant plasmid PTK-EF1α-N6 vector into which the CAR gene was inserted and was capable of expressing CAR was obtained. Its nucleotide sequence is shown in SEQ ID NO. 6.

The virus packaging steps are as follows:

1) Two centrifuge tubes each containing 16 ml of DMEM culture fluid were taken, one of which was added with 960 μg of PEI, and the other tube was added with 320 μg of premixed PTK881 vector plasmid, shaken with vortex, and equilibrated at room temperature for 10 minutes.

2) A 10 ml pipette was used to blow up the culture medium mixed with PEI, and the culture medium mixed with the plasmid was added to PEI drop by drop, and the mixture was incubated at room temperature for 30 minutes.

3) To a T175 bottle, 3 ml of fetal calf serum was added, and the PTK-EF1α-N6 vector plasmid mixed with PEI was added thereto, and then the culture medium in the multi-layer cell culture flask was poured into the T175 bottle, which was shaken up and down and left and right to make the culture medium mixed with the plasmid, and finally the culture medium in the T175 bottle was poured back into the multi-layer cell culture flask. The supernatant was harvested after incubation for 3 days in an at incubator at 37° C. and 5% CO₂. The collected supernatant was centrifuged at 4000 rpm (3000 g) for 30 min to remove 293T cell debris.

4) The lentiviral liquid supernatant was filtered through a 0.22 μm filter, dispensed into 250 ml centrifuge bottles, and centrifuged at 30,000 g for 2.5 hours at 4° C. After centrifugation, the centrifuge bottle was carefully transferred to a biosafety cabinet, and the supernatant was removed by a vacuum pump to leave the precipitate. The T cell culture medium was added with 500 μl/centrifuge bottle. The precipitate was dispersed evenly mixed by blowing with a gun to obtain a lentiviral vector containing the N6-CAR molecule, which was immediately used or dispensed and stored at −80° C.

### Example 3: Preparation of CD8⁺ T Cells (CAR-T Cells) Capable of Expressing a Chimeric Antigen Receptor

### Step 1: Isolation of Patient PBMC Cells

60-80 ml of human peripheral blood samples were collected and shaken while collecting to make the peripheral blood and anticoagulant fully mixed;

the peripheral blood was transferred to a 50 ml centrifuge tube, and diluted with DPBS buffer in a ratio of 1:1 and evenly mixed. The diluted blood sample was slowly added to a centrifuge tube of 15 ml of human lymphocyte separation liquid at room temperature. The method is as follows: the blood sample was pipetted with a 10 ml pipette, the pipette was extended to 0.5 cm above the liquid level of the separation liquid, and the blood sample was naturally slipped onto the separation liquid surface, and then the blood sample was gently added, and the liquid surface was taken carelessly not to be broken.

The mixture was centrifuged for 30 minutes with slow raising speed and slow reducing speed. After centrifugation is completed, the centrifuge tube was clearly layered from bottom to top: red blood cell layer, granulocyte layer, Ficoll layer, mononuclear cell layer and plasma layer. The plasma layer was pipetted to about 5 mm from the white film layer and discarded. All the liquid above the red blood cell layer was carefully pipetted into a centrifuge tube, diluted with PBS, and evenly mixed with the cell suspension in a volume ratio greater than 1:3.

After centrifugation (1600 r/min) for 5 min, the cells were resuspended in PBS and evenly mixed, and a small number of cells were taken for counting.

After centrifugation at 300 g (1200 r/min) for 5 min, the supernatant was sent for sterility testing. Step 2: CD8⁺ T cell sorting

(1) PBMC cells in step 1 were resuspended in 30 ml of normal saline and sampled and counted (after sampling, normal saline was supplemented to 50 ml, the mixture was evenly mixed and centrifuged at 500 g for 10 min at 18° C. with fast raising speed and fast reducing speed, the supernatant was removed). After counting, the cells and buffer were mixed according to 10⁷ cells/80 μL buffer (if the supernatant was not completely removed, it was recommended not to add buffer). The CD8 Microbeads were added according to 10⁷ cells/20 μL of CD8 Microbeads, and the mixture was incubated for 15 min at 4-8° C.

(2) After the incubation is completed, the cells were washed with 1 to 2 ml of buffer per 10⁷ cells, and the mixture was centrifuged at 500 g for 10 min.

(3) Up to 10⁸ cells were resuspended in 500 μL of buffer (if the number of cells was large, more buffer may be used).

(4) A Miltenyi special LS column was placed on a magnetic stand. After washing the LS column with 3 ml of buffer, the cell suspension was added to the LS column and drained. The LS column was washed three times with 3 ml of buffer and each time it was drained. The LS column left the magnetic stand. 5 ml of buffer was added to the LS column and the labeled cells were flushed out with a piston (flushing could be done twice to ensure that the labeled cells can be flushed out).

(5) After the CD8⁺ T cells were flushed out, they were resuspended to 30 ml with normal saline, sampled and counted, and centrifuged at 500 g for 10 min at 18° C. to obtain a cell pellet, which could be used for culture.

### Step 3: Activation of CD8⁺ T Cells

(1) The CD8⁺ T cells in step 2 were counted and added to a culture flask at a density of 2×10⁶/ml, and evenly mixed and placed in a CO₂ incubator and incubated for 2 hours.

(2) The culture flask was taken out and gently shaken to float the suspended cells deposited at the bottom. The culture medium was pipetted into a centrifuge tube. The culture flask was washed with a small amount of culture medium to collect all the suspended cells, which were evenly mixed and counted.

(3) The cell concentration was adjusted according to the cell count, and the cells were inoculated into a culture flask at a concentration of 1.2×10⁶/ml (100 to 120 ml in a T150, 50 to 60 ml in a T75, and 15 to 29 ml in a T25). CD3/CD28 magnetic beads were added thereto at a ratio of cell to magnetic bead 1:3 (before adding, the magnetic beads were washed three times with the culture medium to remove the preservation solution). 100 U/mL IL-2 was added thereto. After evenly mixing, the culture flask was placed in a CO₂ incubator for cultivation, and the cells were collected.

Step 4: CD8⁺ T Cells were Transduced with N6-CAR Molecule to Prepare CAR-T Cells

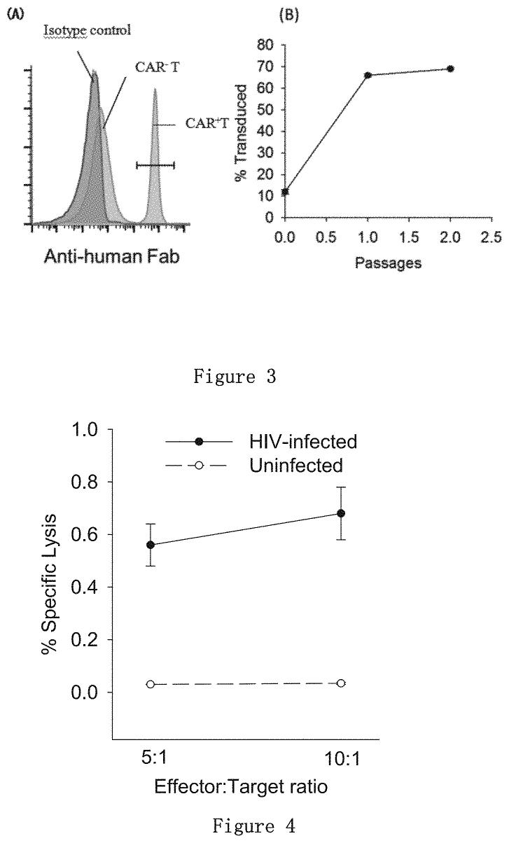

After adding the magnetic beads for 12 hours, an appropriate amount of the CD8⁺ T cell suspension in a good condition in the step 3 was placed in a centrifuge tube and centrifuged at 300 g for 5 minutes, and the supernatant was discarded. The chimeric antigen receptor (CAR) lentiviral vector was added at a ratio of 1×10⁶/ml of cells, while adding Polybrene at a final concentration of 4 μg/ml, and evenly mixed. The cell suspension was incubated in a small volume at 37° C. After incubating for 4 hours, an appropriate amount of T cell complete medium was supplemented for culture. On the third day of cell culture, the cells were counted, and the culture medium was supplemented according to the state and proliferation of the cells. The cell concentration was adjusted to 0.6×10⁶/ml, and 100 U/mL IL-2 was supplemented. On the 5th day of cell culture, the cells were evenly mixed and transferred into a centrifuge tube. The magnetic beads were removed on a magnetic stand. The cells were counted, and supplemented with culture medium and 100 U/mL IL-2. The cell density was adjusted to 0.6×10⁶/ml and the culture was continued. The expression of SCFV was detected by flow cytometry. At the same time, some CD8⁺ T lymphocytes were stimulated by goat anti-human Fab antibody and serially passaged to determine the self-amplification ability of anti-gp120 CAR-transduced CD8⁺ T lymphocytes. The results are shown in FIG. 3.

The results showed that 40% of CD8⁺ T cells expressed CAR molecules after five days of culture of Anti-gp120 CAR virus transduced cells. When the anti-human Fab antibody specifically bound to the CD8⁺ T cell-expressed CAR molecule, it could effectively and dose-dependently activate cell proliferation. As the number of passages increased, the proportion of CAR molecule positive cells population gradually increased.

### Example 4: Detection of Activity of CAR-T Cells in Killing HIV-Infected Cells In Vitro

In order to further detect the function of N6-CAR, N6-CAR-T cells were mixed and cultured with two HIV-1 infected cell lines, H9-NL4-3 and H9-NDK, respectively. The cell killing experiments were performed in 96-well plates in U-bottom. First, the HIV-infected cell line H9 and the negative control cells were labeled with Calcein-AM. 100 μl (contained target cell number: 10⁴) was placed in a 96-well plate, and 100 μl of gradient-diluted CAR-T cells were added to the corresponding 96-well plates to ensure a range of the ratio of effective cells to target cells of 5:1 to 10:1 with a final volume of 200 μl per well. The mixture was centrifuged at 200 g for 30 minutes at room temperature and incubated at 37° C. for 2-3 hours. The supernatant was obtained by centrifugation to measure fluorescence and calculate the percentage of lysis, which was used to determine the cytotoxicity of N6-CAR-T cells against HIV-infected cells. The experimental results are shown in FIG. 4.

The results showed that the N6-CAR-T cells significantly killed the target cell lines infected with two HIV strains in a dose-dependent manner in a range of the ratio of effective cells to target cells of 5:1 to 10:1, but had no obvious killing effect on the control target cells, indicating that the effect of N6-CAR-T cells in killing target cells is specific for HIV-gp120.

### Example 5: Detection of Activity of CAR-T Cells in Inhibiting Viral Replication In Vitro Under Co-Culture Conditions

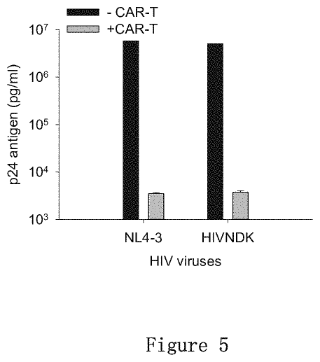

In order to further demonstrate the effectiveness of the N6-CAR-T cells in the clearance of wild-type HIV-1-infected primary CD4+ T cells, the two wild type strains, HIV-1 NL4-3-EGFP and NDK-EGEP, were used to infect CD4+ T lymphocytes isolated from healthy human blood samples, and the cells were changed 3 hours after infection. On the 8th day after infection, the cells were mixed with N6-CAR-modified homologous CD8⁺ T lymphocytes at a ratio of 1:4. The cell killing experiments were performed in a 24-well plate. The target cell number was 10⁶/well and the RMPI 1640 complete medium volume was 500 μl/well. After 48 hours, the ratio of EGFP+CD4+ T lymphocytes was detected by flow cytometry, and the killing effect of the N6-CAR-T cells was verified. The experimental results are shown in FIG. 5.

The results showed that, with the CD8⁺ T cell group modified without CAR molecule as a reference, the N6-CAR-T cell group could clear 99.5% of HIV-1-infected cells and showed a significant killing effect, which fully demonstrated the specificity and efficiency of the N6-CAR-T cells.

### Example 6: Detection of Activity of CAR-T Lymphocytes in Killing HIV Virus-Infected Cells In Vivo

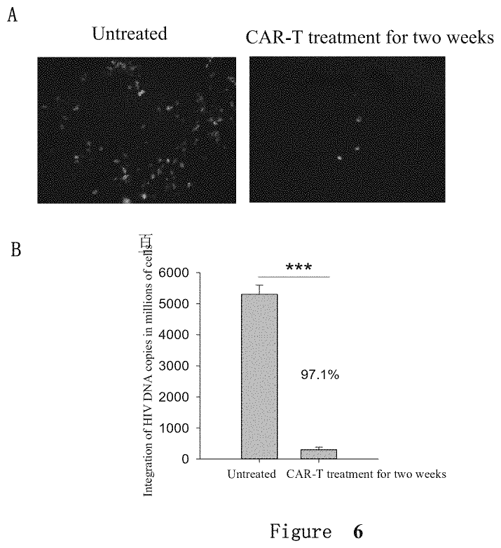

In order to further demonstrate whether the Anti-gp120 CAR-T lymphocytes could clear HIV-infected cells in vivo, the NL4-3-EGFP virus (1×10⁶ pg p24/mouse) carrying the fluorescent gene was intravenously injected into humanized mice BLT, and while infecting the mice, PBMC was isolated from healthy volunteers and then CD8⁺ T cells were isolated, which was transduced with N6-CAR lentivirus, expanded in vitro for 10 days, counted and then resuspended in 500 μl of PBS, and intravenously reinfused at a dose of 1×CD8+ T/kg. Two weeks later, the spleens were collected from the mice and placed in an embedding medium to prepare frozen sections. More than 20 frozen sections with a thickness of 10 μm were prepared, and photographed under a fluorescence confocal microscope. The photographs were quantitatively analyzed using Velocity 5.0 software (FIG. 6A). At the same time, a single cell suspension was prepared using the collected spleen cells, and 5×10⁶ cells were taken to extract genomic DNA using DNAzol. The number of copies of the provirus was quantified using Nested-QPCR to estimate the number of all HIV-infected cells in the body (FIG. 6B).

The results showed that, compared with the control group that did not receive CAR-T cell therapy, the viral protein expression level and the viral genome level were significantly reduced by 97.1%, which confirmed that the CAR-T cells can effectively lyse and clear HIV-infected cells in vivo, laying a theoretical foundation for human clinical testing of the CAR-T cells.

1. A single-chain antibody ScFv wherein the single-chain antibody:
is capable of recognizing gp120 on the surface of an HIV virus-infected cell,
is obtained by tandemly ligating light chain and heavy chain variable regions of an antibody against gp120 on the surface of the HIV virus-infected cell, and
serves as an extracellular binding domain of an entire CAR molecule, and the amino acid sequence thereof is shown in SEQ ID NO. 2.

2. A gene encoding the single-chain antibody ScFv of claim 1, wherein, the nucleotide sequence thereof is shown in SEQ ID NO. 1.

3. A chimeric antigen receptor N6-CAR for treating HIV infection wherein:
the chimeric antigen receptor is obtained by sequentially splicing a signal peptide, the single-chain antibody ScFv of claim 1, CD8 hinge, transmembrane domain of cluster of differentiation CD28-TM and intracellular domain (ICD) thereof, 4-1BB and ζ chain of cluster of differentiation 3 CD3 from N-terminal to C-terminal, and
the amino acid sequence of the obtained chimeric antigen receptor is as shown in SEQ ID NO. 4.

4. The gene encoding the chimeric antigen receptor of claim 3, wherein the nucleotide sequence thereof is shown in SEQ ID NO. 3.

5. An expression vector containing and capable of expressing an encoding gene of a chimeric antigen receptor N6-CAR having the amino acid sequence of SEQ ID NO. 4, wherein:
the vector is a PTK-EF1α-N6 vector obtained by transformation by using a PTK881 vector as a backbone and replacing a CMV promoter with an EF-1α promoter, and
the nucleotide sequence thereof is shown in SEQ ID NO. 5.

6. A genetically modified CD8+ T lymphocyte, being a genetically engineered T-lymphocyte capable of expressing a chimeric antigen receptor obtained by transducing a lentiviral vector of a 293T cell transfected with a PTK-EF1α-N6 expression vector into a CD8+ T lymphocyte.

7. The genetically modified CD8+ T cell of claim 6, being prepared by:
(1) isolating PBMC from peripheral blood and then using magnetic beads for positive screen to obtain CD8+ T cells,
stimulating with anti-CD3/28 magnetic beads (ratio of cell to magnetic bead: 1:3) for 12 hours,
adding a lentivirus recombinant with N6-CAR molecule for infection for 4 hours, and then supplementing fluid (MOI=5),
starting from the third day after virus infection;
counting cells and supplementing the culture medium according to cell state and proliferation,
adjusting the cell concentration to 0.6×106/ml, and
supplementing 100 U/mL IL-2, and
further expanding the cells until the number of cells for reinfusion is met.

8. Use of N6-CAR gene-modified CD8+ T lymphocyte, wherein the genetically modified CD8+ T lymphocyte is used to prepare a live cell drug against HIV infection.


SEQUENCE LISTING (6 sequences)

SEQ ID NO: 1 - DNA, 786 bp, Artificial Sequence
  encoding gene derived from human antibody N6
atgctgctgctggtgaccagcctgctgctgtgcgagctgccccaccccgcccccctgctgatcccccgagcgcacctggtacaatcagggactgcgatgaagaaaccgggggcctcagtaagagtctcctgccagacctctggatacacctttaccgcccacatattattttggttccgacaggcccccgggcgaggacttgagtgggtggggtggatcaagccacaatatggggccgtgaattttggtggtggttttcgggacagggtcacattgactcgagacgtatatagagagattgcgtacatggacatcagaggccttaaacctgacgacacggccgtctattactgtgcgagagaccgttcctatggcgactcctcttgggccttagatgcctggggacagggaacgacggtcgtcgtctccgcgggcggagggggttcaggtggaggaggctctggcggtggcggaagctacatccacgtgacccagtctccgtcctccctgtctgtgtctattggagacagagtcaccatcaattgccagacgagtcagggtgttggcagtgacctacattggtatcaacacaaaccggggagagcccctaaactcttgatccaccatacctcttctgtggaagacggtgtcccctcaagattcagcggctctggatttcacacatcttttaatctgaccatcagcgacctacaggctgacgacattgccacatattactgtcaagttttacaatttttcggccgagggagtcgactccatattaaa

SEQ ID NO: 2 - protein, 262 aa, Artificial Sequence
  chimeric antigen receptor derived from human antibody N6
MLLLVTSLLLCELPHPAFLLIPRAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAGGGGSGGGGSGGGGSYIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIK

SEQ ID NO: 3 - DNA, 1590 bp, Artificial Sequence
  chimeric antigen receptor derived from human antibody N6
atgctgctgctggtgaccagcctgctgctgtgcgagctgccccaccccgcccccctgctgatcccccgagcgcacctggtacaatcagggactgcgatgaagaaaccgggggcctcagtaagagtctcctgccagacctctggatacacctttaccgcccacatattattttggttccgacaggcccccgggcgaggacttgagtgggtggggtggatcaagccacaatatggggccgtgaattttggtggtggttttcgggacagggtcacattgactcgagacgtatatagagagattgcgtacatggacatcagaggccttaaacctgacgacacggccgtctattactgtgcgagagaccgttcctatggcgactcctcttgggccttagatgcctggggacagggaacgacggtcgtcgtctccgcgggcggagggggttcaggtggaggaggctctggcggtggcggaagctacatccacgtgacccagtctccgtcctccctgtctgtgtctattggagacagagtcaccatcaattgccagacgagtcagggtgttggcagtgacctacattggtatcaacacaaaccggggagagcccctaaactcttgatccaccatacctcttctgtggaagacggtgtcccctcaagattcagcggctctggatttcacacatcttttaatctgaccatcagcgacctacaggctgacgacattgccacatattactgtcaagttttacaatttttcggccgagggagtcgactccatattaaaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 4 - protein, 529 aa, Artificial Sequence
  chimeric antigen receptor derived from human antibody N6
MLLLVTSLLLCELPHPAFLLIPRAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAGGGGSGGGGSGGGGSYIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - DNA, 9251 bp, Artificial Sequence
  chimeric antigen receptor derived from human antibody N6
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatctggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcttccatcgaattcctgcagcccgggggatctaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcgccaccacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgagacctggaaaaacatggagcaatcacaagtagcaacacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 6 - DNA, 10841 bp, Artificial Sequence
  chimeric antigen receptor derived from human antibody N6
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatctggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcttccatcgaattcctgcagcccgggggatctaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcgccaccatgctgctgctggtgaccagcctgctgctgtgcgagctgccccaccccgcccccctgctgatcccccgagcgcacctggtacaatcagggactgcgatgaagaaaccgggggcctcagtaagagtctcctgccagacctctggatacacctttaccgcccacatattattttggttccgacaggcccccgggcgaggacttgagtgggtggggtggatcaagccacaatatggggccgtgaattttggtggtggttttcgggacagggtcacattgactcgagacgtatatagagagattgcgtacatggacatcagaggccttaaacctgacgacacggccgtctattactgtgcgagagaccgttcctatggcgactcctcttgggccttagatgcctggggacagggaacgacggtcgtcgtctccgcgggcggagggggttcaggtggaggaggctctggcggtggcggaagctacatccacgtgacccagtctccgtcctccctgtctgtgtctattggagacagagtcaccatcaattgccagacgagtcagggtgttggcagtgacctacattggtatcaacacaaaccggggagagcccctaaactcttgatccaccatacctcttctgtggaagacggtgtcccctcaagattcagcggctctggatttcacacatcttttaatctgaccatcagcgacctacaggctgacgacattgccacatattactgtcaagttttacaatttttcggccgagggagtcgactccatattaaaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgagacctggaaaaacatggagcaatcacaagtagcaacacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc